(19) United States
(12) Patent Application Publication
(10) Pub. No.: US 2002/0193265 A1
(43) Pub. Date: Dec. 19, 2002

(54) Detergent cosmetic compositions containing an anionic surfactant derived from amino acids and salts thereof and a soluble conditioning agent and uses thereof

(76) Inventors: Beatrice Perron, Jouy en Josas (FR); Serge Restle, Saint-Prix (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/108,334

(22) Filed: Mar. 29, 2002

(30) Foreign Application Priority Data
Mar. 30, 2001 (FR) 01 04380

(51) Int. Cl. A61K 7/50

(52) U.S. Cl. 510/130; 510/475; 510/504

(57) ABSTRACT

Novel cosmetic compositions comprising, in a cosmetically acceptable medium, (a) at least one anionic surfactant chosen from N-acylated compounds of mono- and polyamidated polycarboxylic amino acids and the salts of said acids and (b) at least one particular soluble conditioning agent. This combination can impart at least one cosmetic property (such as sleekness, lightness, softness) without keratinous fiber-regreasing phenomenon. These compositions are used, for example, for washing and/or conditioning keratinous materials such as the hair or the skin.

The present invention relates to novel cosmetic compositions comprising, in a cosmetically acceptable medium, at least one anionic surfactant chosen from the N-acylated compounds of mono- and polyamidated polycarboxylic amino acids and the salts of said acids and at least one particular soluble conditioning agent.

The use of detergent compositions (shampoos and shower gels) based essentially on conventional surfactants of the anionic, nonionic and/or amphoteric type, but more particularly of the anionic type, is common for cleansing and/or washing the hair and/or the skin. These compositions are applied to wet hair or skin and the foam generated by massaging or rubbing with the hands allows, after rinsing with water, the removal of the various types of dirt initially present on the hair or the skin.

These base compositions indeed possess good washing power, but the intrinsic cosmetic properties attached to them remain nevertheless fairly weak, in particular because of the fact that the relatively aggressive nature of such a cleansing treatment can cause in the long term damage to the keratinous materials which is marked to a greater or lesser degree, linked in particular to the gradual removal of the lipids or proteins contained in or at the surface thereof.

Accordingly, to improve the cosmetic properties of the above detergent compositions, and more particularly of those that are intended to be applied to sensitive hair (i.e. hair which has become damaged or which has been made fragile especially under the chemical action of atmospheric agents and/or of hair treatments such as permanent waving, dyeing or bleaching), it is now customary to introduce therein additional cosmetic agents called conditioning agents, intended mainly to repair or limit the harmful or undesirable effects induced by the various treatments or attacks to which the hair fibres are subjected more or less repeatedly. These conditioning agents can of course also improve the cosmetic behaviour of natural hair.

The conditioning agents most commonly used to date in shampoos are cationic polymers, silicones and/or silicone-based derivatives, which indeed confer on washed, dry or wet hair greatly improved ease of disentanglement, softness and sleekness compared to what may be obtained with the corresponding cleansing compositions not containing them.

However, in spite of the progress recently made in the field of shampoos based on cationic polymers and/or silicone, the latter are not completely truly satisfactory, such that a high demand currently still exists in relation to being able to have novel products exhibiting better performance at the level of at least one of the cosmetic properties mentioned above.

The N-acylated anionic surfactants of mono- and polyamidated polycarboxylic amino acids and their salts have already been recommended in detergent cosmetic compositions. They have been described, for example, in patent application WO 97/03171, the disclosure of which is hereby incorporated by reference.

The washing compositions for the hair using these surfactants alone may not lead to completely satisfactory cosmetic properties.

The aim of the invention is therefore to provide detergent cosmetic compositions having at least one improved cosmetic property, such as disentanglement, sleekness and/or softness of the hair.

However, the inventors have now found that the combination of particular soluble conditioning agents and a particular anionic surfactant and their salts makes it possible to achieve at least one of these aims.

These novel compositions can make it possible to better deposit these conditioning agents on keratinous materials (especially the hair) than a composition containing conventional anionic surfactants such as the salts of N-cocoylglutamic acid.

The compositions in accordance with the invention confer on the keratinous materials, in particular the hair, a remarkable treatment effect which manifests itself in particular by at least one of the properties of lightness, sleekness, softness, suppleness, and ease of disentanglement with minimal sensation of a charged feel.

The subject of the invention is thus a detergent cosmetic composition, characterized in that it comprises, in a cosmetically acceptable medium, at least one anionic surfactant chosen from the N-acylated compounds of mono- and polyamidated polycarboxylic amino acids and the salts of said acids and at least one soluble conditioning agent chosen from:

I) anionic polymers,

II) nonionic polymers,

III) cationic polymers, such as cationic proteins and hydrolysates of cationic proteins

IV) amphoteric polymers, and

V) cationic surfactants chosen from quaternary ammonium salts comprising at least one amide functional group, quaternary ammonium salts of imidazoline, quaternary diammonium salts, quaternary ammonium salts containing at least one ester functional group.

Another subject of the invention relates to the use of at least one anionic surfactant chosen from the N-acylated compounds of mono- and polyamidated polycarboxylic amino acids and the salts of said acids in, or for the manufacture of a cosmetic composition comprising at least one soluble conditioning agent as defined above.

Another subject of the invention relates to a method for treating a keratinous material, such as the hair, comprising applying to said material an effective amount of a cosmetic composition according to the invention.

A subject of the invention is also the use of a composition according to the invention in order to increase at least one of disentanglement and sleeking of the hair, in order to impart at least one of volume, lightness, softness, suppleness and manageability on the hair.

According to the present invention, the expression keratinous material is understood to encompass the hair, the eyelashes, the eyebrows, the skin, the mucous membranes or the scalp, and more particularly the hair.

Various aspects of the invention will now be presented in detail. All the meanings and definitions of the compounds used in the present invention given below are valid for all aspects of the invention.

N-Acylated Anionic Surfactants of Mono and Polyamidated Polycarboxylic Amino Acids

The N-acylated anionic surfactants of mono- and polyamidated polycarboxylic amino acids and their salts may have the following formula (I):

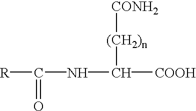

in which:

R is chosen from linear and branched, saturated and unsaturated, hydrocarbon radicals comprising from 5 to 29 carbon atoms. For example, R is chosen from mono- and polyunsaturated alkyl and alkenyl radicals comprising from 5 to 29 carbon atoms and as a further example from 7 to 22 carbon atoms,

n is an integer chosen from 1 and 2.

An additional example would be N-acylated compounds of mono- and polyamidated polycarboxylic amino acids of formula (I) in which R is chosen from linear, branched, and saturated alkyl radicals comprising from 7 to 29 carbon atoms, such as from 7 to 22 carbon atoms.

The salts of the compounds of formula (I) may be salts of alkali metals (for example sodium and potassium), of alkaline-earth metals (which may be, for example, calcium and magnesium), salts of aqueous ammonia, salts of amines such as those of monoethanolamine, diethanolamine, triethanolamine, 3-amino-1,2-propanediol, and ammonium salts derived from basic amino acids such as lysine, arginine, sarcosine, ornithine, citrulline.

Among the surfactants of formula (I), there may be mentioned for example the salts of N-cocoylglutamine and in particular the triethanolamine salt of N-cocoylglutamine such as the product marketed under the name FOAM UP DOUCE GM by the company KYOWA HAKKO.

According to the invention, the anionic surfactant(s) chosen from the N-acylated compounds of mono- and polyamidated polycarboxylic amino acids and their salts may, for example, represent from 1% to 30% by weight, such as from 3% to 15% by weight relative to the total weight of the final composition.

The expression water-soluble conditioning agents is understood to mean conditioning agents soluble in water at a concentration greater than or equal to 0.1% by weight in water at 25° C., that is to say that they form a macroscopically isotropic transparent solution, meaning that the solution is homogeneous to the naked eye and, to the naked eye, has properties that do not vary with direction.

Anionic Polymers

The anionic polymers generally used are polymers comprising groups derived from an acid chosen from carboxylic, sulphonic and phosphoric acids and have a weight-average molecular weight of from about 500 to 5 000 000 determined, for example, by gel permeation chromatography.

1) The carboxylic groups can be provided by monomers chosen from unsaturated monocarboxylic and dicarboxylic acid monomers, such as those corresponding to the formula:

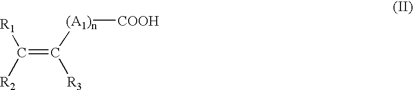

in which:

n is an integer from 0 to 10,

A₁ is chosen from a methylene group wherein, when n is greater than 1, at least one of said methylene groups can be bonded to a hetero atom, such as oxygen and sulphur,

R₁ is chosen from hydrogen, phenyl groups and benzyl groups,

R₂ is chosen from hydrogen, lower alkyls, and carboxyl groups,

R₃ is chosen from hydrogen, lower alkyls, —CH₂—COOH, phenyl groups, and benzyl groups.

In the abovementioned formula, a lower alkyl group denotes C₁-C₈ alkyls, such as alkyl groups having 1 to 4 carbon atoms, for example, methyl and ethyl.

Exemplary anionic fixing polymers comprising carboxylic groups include:

A) homo- and copolymers derived from acrylic and methacrylic acid monomers, and salts thereof, such as products sold under the names Versicol® E or K by the company Allied Colloid, and Ultrahold® by the company BASF; and copolymers derived from monomers chosen from acrylic acids and acrylamides, such as those sold in the form of their sodium salt under the names RETEN 421, 423, and 425 by the company HERCULES, and sodium salts of polyhydroxycarboxylic acids.

B) copolymers derived from (a) monomers chosen from acrylic and methacrylic acid, and (b) monoethylenic monomers such as ethylene, styrene, vinyl esters, acrylic and methacrylic acid esters. These copolymers can be optionally grafted onto a polyalkylene glycol such as polyethylene glycol, and the graft polymers can be optionally crosslinked. Exemplary polymers are described in French Patent No. 1 222 944 and German patent application No. 2 330 956, the disclosures of which are specifically incorporated by reference herein. Copolymers containing an optionally N-alkylated and/or hydroxyalkylated acrylamide unit in their chain also fall within this class of polymers, and are described in Luxembourg patent application Nos. 75370 and 75371, the disclosures of which are specifically incorporated by reference herein, such as the copolymers sold under the name QUADRAMER by AMERICAN CYANAMID. Mention may also be made of copolymers derived from acrylic acid and C₁-C₄ alkyl methacrylates, and terpolymers derived from vinylpyrrolidone, acrylic acid and C₁-C₂₀ alkyl methacrylates, for example lauryl, such as the product sold by the company ISP under the name ACRYLIDONE® LM and methacrylic acid/ethyl acrylate/tert-butyl acrylate terpolymers such as the product sold under the name LUVIMER® 100 P by the company BASF.

C) copolymers derived from crotonic acid, such as those containing vinyl acetate and propionate units, and optionally other monomers such as allylic esters, methallylic esters, and vinyl ethers and vinyl esters of linear and branched saturated carboxylic acids with a long hydrocarbon-based chain such as those containing at least 5 carbon atoms. These polymers can be grafted and crosslinked. The copolymers can also alternatively be derived from another monomer chosen from vinyl, allylic and methallylic esters of α- and β-cyclic carboxylic acids. Such polymers are described, inter alia, in French patent Nos. 1 222 944, 1 580 545, 2 265 782, 2 265 781, 1 564 110, and 2 439 798, the disclosures of which are specifically incorporated by reference herein. A commercial product falling into this class is the resin 28-29-30, 26-13-14, and 28-13-10, sold by the company National Starch.

D) copolymers derived from C₄-C₈ monounsaturated carboxylic acids and anhydrides, the copolymers being chosen from:

copolymers derived from (i) at least one monomer chosen from maleic, fumaric, and itaconic acids and anhydrides, and (ii) at least one monomer chosen from vinyl esters, vinyl ethers, vinyl halides, phenylvinyl derivatives, acrylic acids and its esters. The anhydride functions of these copolymers can be monoesterified or monoamidated. Exemplary polymers are described in U.S. Pat. Nos. 2,047,398, 2,723,248, and 2,102,113, and GB patent No. 839 805, the disclosures of which are specifically incorporated by reference herein. Exemplary commercial products include those sold under the names Gantrez® AN and ES by the company ISP,

copolymers derived from (i) at least one monomer chosen from maleic, citraconic, and itaconic anhydrides, and (ii) at least one monomer chosen from allylic and methallylic esters optionally containing at least one group chosen from acrylamides, methacrylamides, a-olefin groups, acrylic esters, methacrylic esters, acrylic acids, methacrylic acids, and vinylpyrrolidone, wherein the monomeric residues are present in the chains of the copolymers. The anhydride functions of these copolymers can be monoesterified or monoamidated. Exemplary polymers are described in French patent Nos. 2 350 384 and 2 357 241, the disclosures of which are specifically incorporated by reference herein.

E) polyacrylamides containing carboxylate groups.

2) The polymers comprising sulphonic groups are chosen from polymers comprising vinylsulphonic, styrenesulphonic, naphthalenesulphonic and acrylamidoalkylsulphonic units.

These polymers may be chosen from:

the salts of polyinylsulphonic acid having a weight-average molecular weight of from about 1000 to 100,000 as well as the copolymers derived from an unsaturated comonomer chosen from acrylic and methacrylic acids and their esters, acrylamide and their derivatives, vinyl ethers and vinylpyrrolidone;

the salts of polystyrenesulphonic acid, the sodium salts having a weight-average molecular weight of about 500,000 and about 100,000 sold respectively under the names Flexan 500 and Flexan 130 by National Starch. These compounds are described in Patent FR 2,198,719, the disclosure of which is hereby incorporated by reference; and

the salts of polyacrylamidesulphonic acids, for example, the salts mentioned in U.S. Pat. No. 4,128,631, the disclosure of which is hereby incorporated by reference, and, as a further example, polyacrylamidoethylpropanesulphonic acid sold under the name COSMEDIA POLYMER HSP 1180 by Henkel.

According to the invention, the anionic film-forming polymers are chosen from acrylic acid copolymers such as the terpolymer acrylic acid/ethyl acrylate/N-tert-butylacrylamide sold under the name ULTRAHOLD STRONG by the company BASF, copolymers derived from crotonic acid such as the terpolymers vinyl acetate/vinyl tert-butylbenzoate/crotonic acid and the terpolymers crotonic acid/vinyl acetate/vinyl neododecanoate sold under the name Resine 28-29-30 by the company NATIONAL STARCH, polymers derived from at least one monomer chosen from itaconic, fumaric and maleic acids and anhydrides and at least one monomer chosen from vinyl esters, vinyl ethers, vinyl halides, phenylvinyl derivatives, acrylic acid and its esters, such as the monoesterified maleic anhydride and methylvinyl ether copolymer sold under the name GANTREZ ES 425 by the company ISP; copolymers of methacrylic acid and methyl methacrylate sold under the name EUDRAGIT L by the company ROHM PHARMA; copolymers of methacrylic acid/ethyl acrylate sold under the names LUVIMER MAEX and MAE by the company BASF; and copolymers of vinyl acetate/crotonic acid sold under the name LUVISET CA 66 by the company BASF and the copolymer of vinyl acetatecrotonic acid grafted with polyethylene glycol under the name ARISTOFLEX A by the company BASF.

Additional examples of anionic film-forming polymers include those chosen from monoesterified maleic anhydride/methylvinyl ether copolymers sold under the name GANTREZ ES 425 by the company ISP; terpolymer acrylic acid/ethyl acrylate/N-tert-butylacrylamides sold under the name ULTRAHOLD STRONG by the company BASF; copolymers of methacrylic acid and of methyl methacrylate sold under the name EUDRAGIT L by the company ROHM PHARMA; terpolymers of vinyl acetate/vinyl tert-butylbenzoate/crotonic acid and terpolymers of crotonic acid/vinyl acetate/vinyl neododecanoate sold under the name Resine 28-29-30 by the company NATIONAL STARCH; copolymers of methacrylic acid and ethyl acrylate sold under the name LUVIMER MAEX and MAE by the company BASF; and terpolymers of vinylpyrrolidone/acrylic acid/lauryl methacrylate sold under the name ACRYLIDONE LM by the company ISP.

Amphoteric Polymers

The amphoteric polymers can be chosen from polymers comprising K and M units distributed randomly in the polymer chain, in which K is derived from a monomer containing at least one basic nitrogen atom, and M is derived from an acid monomer containing at least one group chosen from carboxylic and sulphonic groups. Alternatively K and M can be derived from zwitterionic monomers chosen from carboxybetaines and sulphobetaines.

K and M can also comprise a cationic polymer chain containing groups chosen from primary, secondary, tertiary and quaternary amine groups, in which at least one of the amine groups bears a groups chosen from carboxylic and sulphonic groups connected via a hydrocarbon-based group. Alternatively, K and M can form part of a chain of a polymer containing an α,β-dicarboxylic ethylene unit in which one of the carboxylic groups has been made to react with a polyamine containing at least one group chosen from primary and secondary amine groups.

Examples of the amphoteric polymers corresponding to the definition above include those chosen from:

(1) polymers resulting from the copolymerization of (i) monomers derived from a vinyl compound bearing a carboxylic group, such as monomers chosen from acrylic acids, methacrylic acids, maleic acids, α-chloroacrylic acids, and (ii) basic monomers derived from a substituted vinyl compound comprising at least one basic atom, such as those monomers chosen from dialkylaminoalkyl methacrylates and acrylates, and dialkylaminoalkylmethacrylamides and dialkylaminoalkylacrylamides. Exemplary polymers are described in U.S. Pat. No. 3,836,537, the disclosure of which is specifically incorporated by reference herein. Mention may also be made of the sodium acrylate/acrylamidopropyltrimethylammonium chloride copolymer sold under the name Polyquart KE 3033 by the company HENKELI.

The vinyl compound may also be a dialkyldiallylammonium salt such as diethyldiallylammonium chloride. Exemplary copolymers derived from acrylic acid and the latter monomer are sold under the names MERQUAT 280, MERQUAT 295 and MERQUAT Plus 3330 by the company Calgon.

(2) polymers derived from:

a) at least one monomer chosen from acrylamides and methacrylamides substituted on the nitrogen atom with an alkyl group,

b) at least one acidic comonomer containing at least one reactive carboxylic group, and

c) at least one basic comonomer, such as esters comprising at least one substituent chosen from primary, secondary, tertiary and quaternary amine substituents of acrylic and methacrylic acids, and the product of quaternization of dimethylaminoethyl methacrylate with a sulphate chosen from dimethyl and diethyl sulphates.

Exemplary N-substituted acrylamides and methacrylamides include those compounds in which the alkyl groups contain from 2 to 12 carbon atoms, such as N-ethylacrylamide, N-tert-butylacrylamide, N-tert-octylacrylamide, N-octylacrylamide, N-decylacrylamide, N-dodecylacrylamide and the corresponding methacrylamides.

Exemplary acidic comonomers include those chosen from acrylic acid, methacrylic acid, crotonic acid, itaconic acid, maleic acid, fumaric acid, alkyl monoesters, having 1 to 4 carbon atoms, of maleic and fumaric acids, and alkyl monoesters, having 1 to 4 carbon atoms, of maleic and fumaric anhydrides.

Exemplary basic comonomers include those chosen from aminoethyl methacrylate, butylaminoethyl methacrylate, N,N′-dimethylaminoethyl methacrylate, and N-tert-butylaminoethyl methacrylate.

The copolymers whose CTFA (4th edition, 1991) name is octylacrylamide/acrylates/butylaminoethyl methacrylate copolymer, such as the products sold under the name Amphomer® and Lovocryl® 47 by the company National Starch, can be used.

(3) crosslinked and acylated polyamino amides chosen from those partially and totally derived from polyamino amides of the formula:

(CO—R₁₀—CO—Z) (II)

in which R₁₀ is chosen from divalent groups derived from saturated dicarboxylic acids, mono- and dicarboxylic aliphatic acids containing an ethylenic double bond, esters of these acids of a lower alkanol having 1 to 6 carbon atoms inclusive, and a group derived from the addition of any one of the said acids to an amine chosen from bis(primary) and bis(secondary) amines, and

Z is chosen from a group derived from bis(primary), mono- and bis(secondary) polyalkylene-polyamines. Z can, for example, be chosen from:

a) in proportions of from 60 to 100 mol %, the groups

—NH[(CH₂)ₓ—NH]ₚ (IV)

where x=2 and p=2 or 3, or alternatively x=3 and p=2, this group being derived from amines chosen from diethylenetriamines, triethylenetetraamines, and dipropylenetriamines;

b) in proportions of from 0 to 40 mol %, groups chosen from the group (IV) above in which x=2 and p=1 and which is derived from ethylenediamine and the group derived from piperazine:

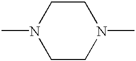

c) in proportions of from 0 to 20 mol %, the —NH—(CH₂)₆—NH— group derived from hexamethylenediamine. These polyamino amines can be crosslinked by reaction addition of a difunctional crosslinking agent chosen from epihalohydrines, diepoxides, dianhydrides and bis-unsaturated derivatives, by using from 0.025 to 0.35 mol of crosslinking agent per amine group of the polyamino amide and can be alkylated by the action of acrylic acid, chloroacetic acid, alkane sultones, and salts thereof.

Exemplary saturated carboxylic acids are chosen from acids having 6 to 10 carbon atoms, such as adipic acid, 2,2,4-trimethyladipic acid, 2,4,4-trimethyladipic acid, terephthalic acid, and acids containing an ethylenic double bond such as, for example, acrylic acid, methacrylic acid and itaconic acid.

The alkane sultones used in the alkylation can be chosen from propane sultone and butane sultone. The salts of the acylating agents can be chosen from sodium and potassium salts.

(4) polymers containing zwitterionic units derived from at least one monomer of formula:

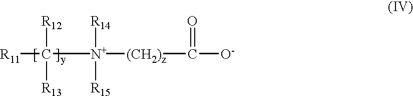

in which:

R₁₁ is chosen from polymerizable unsaturated groups, such as acrylates, methacrylates, acrylamides and methacrylamides,

y and z are chosen from an integer from 1 to 3,

R₁₂ and R₁₃ are chosen from hydrogen, methyl, ethyl and propyl groups,

R₁₄ and R₁₅ are chosen from hydrogen and alkyl groups such that the sum of the carbon atoms in R₁₄ and R₁₅ does not exceed 10.

The polymers comprising such units can also contain units derived from non-zwitterionic monomers such as those monomers chosen from dimethyl and diethylaminoethyl acrylates and methacrylates, alkyl acrylates and methacrylates, acrylamides and methacrylamides, and vinyl acetate.

By way of example, mention may be made of the copolymers of butyl methacrylate/N,N-dimethylcarboxyaminoethyl methacrylate sold under the name DIAFORMER Z301 by the company SANDOZ.

(5) polymers comprising chitosan containing monomeric units corresponding to the following formulae:

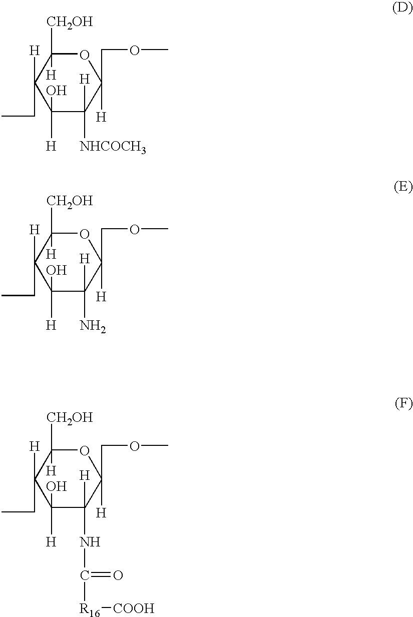

the unit (D) being present in proportions of between 0 and 30% inclusive, the unit (E) in proportions of between 5 and 50% inclusive and the unit (F) in proportions of between 30 and 90% inclusive, it being understood that, in this unit (F), R₁₆ is a group of formula:

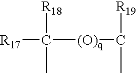

in which:

if q=0, then R₁₇, R₁₈ and R₁₉, which may be identical or different, are chosen from hydrogen, methyl, hydroxyl, acetoxy, amino, monoalkylamines and dialkylamines, the alkyl portion of which may be optionally interrupted by at least one nitrogen atom and/or optionally substituted with at least one group chosen from amine, hydroxyl, carboxyl, alkylthio and sulphonic groups, and alkylthio groups in which the alkyl group bears an amino group; at least one of the groups R₁₇, R₁₈ and R₁₉ being, in this case, hydrogen;

or, if q =1, R₁₇, R₁₈ and R₁₉, which may be identical or different, are chosen from hydrogen, and

acid and base addition salts of said polymers comprising said chitosan containing monomeric units.

(6) polymers corresponding to the general formula (V), examples of which are described in French patent 1 400 366, the disclosure of which is specifically incorporated by reference herein:

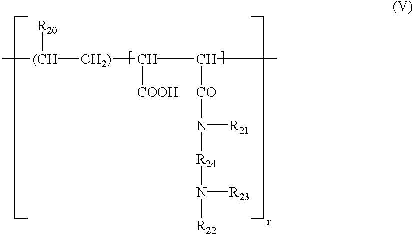

in which:

R₂₀ is chosen from hydrogen, C₁₆ alkoxy, such as CH₃O and CH₃CH₂O, and phenyl,

R₂₁ is chosen from hydrogen and C₁-C₆ alkyl groups, such as methyl and ethyl,

R₂₂ is chosen from hydrogen and C₁-C₆ alkyl groups, such as methyl and ethyl,

R₂₃ is chosen from C₁-C₆ alkyl groups, such as methyl and ethyl, and groups corresponding to the formula: —R₂₄—N(R₂₂)₂, wherein R₂₄ is chosen from —CH₂—CH₂—, —CH₂—CH₂—CH₂—, and —CH₂—CH(CH₃)—, and R₂₂ is defined as above, and

r is chosen such that the number-average molecular weight of said polymer ranges from 500 to 6,000,000, such as from 1000 to 1,000,000.

(7) polymers derived from the N-carboxyalkylation of chitosan, such as N-carboxymethylchitosan and N-carboxybutylchitosan, which is sold under the name “EVALSAN” by the company JAN DEKKER.

(8) amphoteric polymers of the type —D—X—D—X chosen from:

a) polymers obtained by the action of a reactant, chosen from chloroacetic acid and sodium chloroacetate, on compounds comprising at least one unit of formula:

—D—X—D—X—D— (VI)

wherein D is

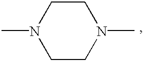

and

X is chosen from the symbols E and E′, wherein E and E′, which may be identical or different, are each chosen from divalent alkylene groups comprising at least one chain chosen from linear and branched chains comprising up to 7 carbon atoms in the principal chain, wherein said divalent alkylene groups are optionally substituted with at least one hydroxyl group. E and E′ can additionally comprise: a hetero atom chosen from oxygen, nitrogen, sulphur; and 1 to 3 rings chosen from aromatic and heterocyclic rings. The oxygen, nitrogen and sulphur atoms can be present in the form of at least one group chosen from ether, thioether, sulphoxide, sulphone, sulphonium, alkylamine and alkenylamine groups, hydroxyl, benzylamine, amine oxide, quaternary ammonium, amide, imide, alcohol, ester and urethane groups.

b) polymers of formula:

—D—X—D—X— (VI′)

wherein D is

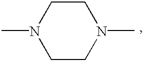

and

X is chosen from the symbols E and E′ and wherein at least one X is chosen from E′;

E is chosen from divalent alkylene groups comprising at least one chain chosen from linear and branched chains comprising up to 7 carbon atoms in the principal chain, wherein said divalent alkylene groups are optionally substituted with at least one hydroxyl group. E can additionally comprise: a hetero atom chosen from oxygen, nitrogen, sulphur; and 1 to 3 rings chosen from aromatic and heterocyclic rings. The oxygen, nitrogen and sulphur atoms can be present in the form of at least one group chosen from ether, thioether, sulphoxide, sulphone, sulphonium, alkylamine and alkenylamine groups, hydroxyl, benzylamine, amine oxide, quaternary ammonium, amide, imide, alcohol, ester and urethane groups, and

E′ is chosen from divalent alkylene groups comprising at least one chain chosen from linear and branched chains comprising up to 7 carbon atoms in the principal chain, wherein said divalent alkylene groups are optionally substituted with at least one hydroxyl group. E″ can comprise at least one nitrogen atom substituted with an alkyl chain which can be interrupted by an oxygen atom and, wherein said alkyl chain comprises at least one functional group chosen from carboxyl functional groups and hydroxyl functional groups, and wherein said at least one alkyl chain is betainized by reaction with a reactant chosen from chloroacetic acid and sodium chloroacetate.

(9) (C₁-C₅)alkyl vinyl ether/maleic anhydride copolymers partially modified by reactions chosen from semiamidation with an N,N-dialkylaminoalkylamine such as N,N-dimethylaminopropylamine, and from semiesterification with an N,N-dialkylaminoalkynol. These copolymers can also contain other vinyl comonomers such as vinylcaprolactam. As a further example, the amphoteric polymers are chosen from group (1) above.

Nonionic Polymers

The nonionic polymers can be chosen from:

vinylpyrrolidone homopolymers;

vinylpyrrolidone and vinyl acetate copolymers;

polyalkyloxazolines such as the polyethyl-oxazolines sold by the company DOW CHEMICAL under the names PEOX 50,000, PEOX 200,000 and PEOX 500,000;

vinyl acetate homopolymers such as the product sold under the name APPRETAN EM by the company HOECHST and the product sold under the name RHODOPAS A 012 by the company RHONE POULENC;

acrylic ester and vinyl acetate copolymers such as the product sold under the name RHODOPAS AD 310 by the company RHONE POULENC;

ethylene and vinyl acetate copolymers such as the product sold under the name APPRETAN TV by the company HOECHST;

copolymers of vinyl acetate and maleic ester, for example, dibutyl maleate, such as the product sold under the name APPRETAN MB EXTRA by the company HOECHST;

maleic anhydride and polyethylene copolymers;

homopolymers of alkyl acrylates and the homopolymers of alkyl methacrylates such as the product sold under the name MICROPEARL RQ 750 by the company MATSUMOTO and the product sold under the name LUHYDRAN A 848 S by the company BASF;

copolymers of acrylic esters, for example, copolymers of alkyl acrylates and alkyl methacrylates such as the products sold by the company ROHM & HMS under the names PRIMAL AC-261 K and EUDRAGIT NE 30 D, the products sold by the company BASF under the names ACRONAL 601, LUHYDRAN LR 8833 and 8845, and the products sold by the company HOECHST under the names APPRETAN N 9213 and N9212;

copolymers of acrylonitrile and of a nonionic monomer chosen from butadiene and alkyl (meth)acrylates such as the products sold under the name NIPOL LX 531 B by the company NIPPON ZEON and the products sold under the name CJ 0601 B by the company ROHM & HMS;

polyurethanes such as the products sold under the names ACRYSOL RM 1020 and ACRYSOL RM 2020 by the company ROHM & HAAS and the products sold under the names URAFLEX XP 401 UZ and URAFLEX XP 402 UZ by the company DSM RESINS;

copolymers of alkyl acrylate and urethane such as the product sold under the number 8538-33 by the company NATIONAL STARCH;

polyamides such as the product sold under the name ESTAPOR LO 11 by the company RHONE POULENC; and

guar gums chosen from chemically modified nonionic guar gums and unmodified nonionic guar gums.

The alkyl groups of the nonionic polymers have from 1 to 6 carbon atoms unless otherwise stated.

Examples of the unmodified nonionic guar gums include the products sold under the name VIDOGUM GH 175 by the company UNIPECTINE and the products sold under the name JAGUAR C by the company MEYHALL.

Examples of the modified nonionic guar gums include nonionic guar gums modified with C₁-C₆ hydroxyalkyl groups such as hydroxymethyl, hydroxyethyl, hydroxypropyl and hydroxybutyl groups.

These guar gums are well known in the state of the art and may, for example, be prepared by reacting corresponding alkene oxides, such as for example propylene oxides, with the guar gum so as to obtain a guar gum modified with hydroxypropyl groups.

Examples of nonionic guar gums optionally modified with hydroxyalkyl groups include the products sold under the trade names JAGUAR HP8, JAGUAR HP60, JAGUAR HP120, JAGUAR DC 293 and JAGUAR HP 105 by the company MEYHALL and the products sold under the name GALACTASOL 4H₄FD2 by the company AQUALON.

The polyurethanes are chosen from functionalized polyurethanes and non-funtionalized polyurethanes. Examples include the polyurethanes described in Patents EP 0,751,162, EP 0,637,600, FR 2,743,297 and EP 0,648,485, of which L'Oreal, S. A., is the proprietor, as well as Patents EP 0,656,021 and WO 94/03510 by the company BASF and EP 0,619,111 by the company National Starch. The disclosures of these patents are hereby incorporated by reference.

Cationic Polymers

The conditioning agents of the cationic polymer type which can be used in accordance with the present invention may be chosen from all those already known per se as improving the cosmetic properties of hair treated with detergent compositions, such as those described in Patent Application EP—A-0,337,354 and in French Patent Applications FR—A-2,270,846, 2,383,660, 2,598,611, 2,470,596 and 2,519,863, the disclosures of which are hereby incorporated by reference.

For the purposes of the present invention, the expression “cationic polymer” denotes any polymer containing cationic groups and/or groups ionizable into cationic groups. Examples include cationic polymers chosen from cationic polymers comprising at least one group chosen from primary, secondary, tertiary and quaternary amine groups in the principal polymer chain and cationic polymers comprising at least one group chosen from primary, secondary, tertiary and quaternary amine groups in a side substituent directly linked to the principal polymer chain.

Exemplary cationic polymers have a number-average molecular mass of from 500 to 5×10⁶ approximately, such as from 10³ to 3×10⁶ approximately.

As a further example of cationic polymers, mention may be made of polyamine polymers, polyaminoamide polymers, and quaternary polyammonium polymers. These polymers are known products, and examples include those polymers described in French Patents No. 2,505,348 and 2,542,997, the disclosures of which are hereby incorporated by reference.

Further examples of polyamine polymers, polyaminoamide polymers, and quaternary polyammonium polymers include the polymers chosen from:

(1) Homopolymers and copolymers derived from monomers chosen from acrylic and methacrylic esters and amides and comprising at least one of the units of the following formulae:

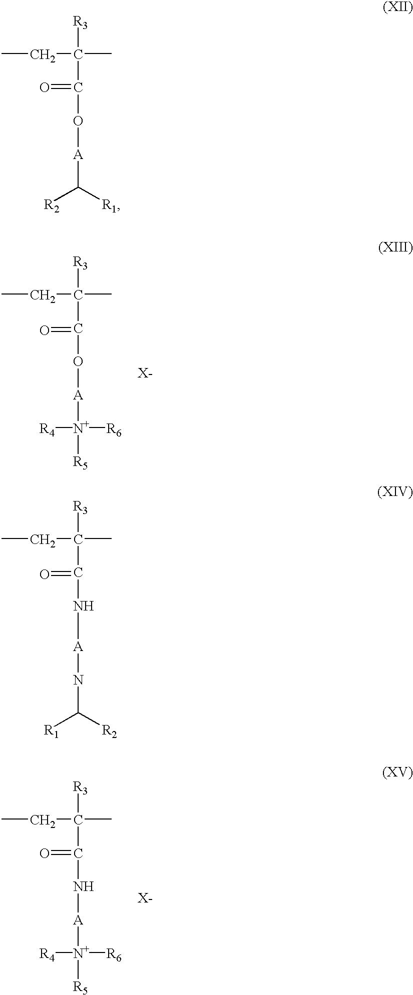

in which:

R₁ and R₂, which are identical or different, are chosen from hydrogen and alkyl groups comprising 1 to 6 carbon atoms, such as methyl and ethyl;

R₃, which are identical or different, are chosen from a hydrogen atom and a CH₃ radical;

A, which are identical or different, are chosen from linear and branched alkyl groups of 1 to 6 carbon atoms, such as 2 or 3 carbon atoms, and hydroxyalkyl groups of 1 to 4 carbon atoms;

R₄, R₅, R₆, which are identical or different, are chosen from alkyl groups comprising from 1 to 18 carbon atoms, such as from 1 to 6 carbon atoms, and benzyl radicals; and

is an anion chosen from anions derived from inorganic acids and anions derived from organic acids, for example, methosulphate anions and halides such as chloride or bromide.

The copolymers of the family (1) may contain, in addition, one or more units derived from comonomers which may be chosen from the family of acrylamides, methacrylamides, diacetone acrylamides, acrylamides and methacrylamides substituted on the nitrogen with a substituent chosen from (C₁-C₄)alkyls, acrylic and methacrylic acids and esters thereof, vinylactams such as vinylpyrrolidone and vinylcaprolactam, and vinyl esters.

Thus, examples of the copolymers of family (1), include:

copolymers of acrylamide and dimethylamino-ethyl methacrylate quaternized with dimethyl sulphate or with a dimethyl halide such as the product sold under the name HERCOFLOC by the company HERCULES;

copolymers of acrylamide and methacryloyloxy-ethyltrimethylammonium chloride described, for example, in Patent Application EP—A-080976, the disclosure of which is herein incorporated by reference, and sold under the name BINA QUAT P 100 by the company CIBA GEIGY;

copolymers of acrylamide and methacryloyloxy-ethyltrimethylammonium methosulphate sold under the name RETEN by the company HERCULES;

copolymers of vinylpyrrolidone/dialkylaminoalkyl acrylate and vinylpyrrolidone/methacrylate copolymers, quaternized and otherwise, for example, the products sold under the name “GAFQUAT” by the company ISP, such as “GAFQUAT 734” and “GAFQUAT 755,” and products called “COPOLYMER 845, 958 and 937”. These polymers are described in detail in French Patents 2,077,143 and 2,393,573, the disclosures of which are hereby incorporated by reference;

dimethylaminoethyl methacrylate/vinylcapro-lactam/vinylpyrrolidone terpolymers such as the product sold under the name GAFFIX VC 713 by the company ISP;

vinylpyrrolidone/methacrylamidopropyldimethyl-amine copolymers sold under the name STYLEZE CC 10 by ISP; and

quaternized vinylpyrrolidone/dimethyl-aminopropyl methacrylamide copolymers such as the product sold under the name “GAFQUAT HS 100” by the company ISP.

2) Cationic polysaccharides such as cationic celluloses and galactomannan gums. Further examples of cationic polysaccharides include derivatives of cellulose ethers comprising quaternary ammonium groups, cationic copolymers of cellulose, derivatives of cellulose grafted with a water-soluble quaternary ammonium monomer and cationic galactomannan gums.

Examples of cellulose ether derivatives comprising quaternary ammonium groups include those cellulose ether derivatives that are described in French Patent 1,492,597, the disclosure of which is hereby incorporated by reference, and sold, for example, under the names “JR” (JR 400, JR 125, JR 30M) or “LR” (LR 400, LR 30M) by the company Union Carbide Corporation. These polymers are also defined in the CTFA dictionary as hydroxyethyl cellulose quaternary ammoniums which have reacted with an epoxide substituted by a trimethyl-ammonium group.

Cationic cellulose copolymers and cellulose derivatives grafted with a quaternary ammonium water-soluble monomer are described in U.S. Pat. No. 4,131,576, the disclosure of which is hereby incorporated by reference. Examples include hydroxyalkyl celluloses such as hydroxymethyl, hydroxyethyl and hydroxypropyl celluloses and grafted hydroxyalkyl celluloses, grafted, for example, with at least one salt chosen from methacryloylethyltrimethylammonium, methacrylamidopropyltrimethylammonium and dimethyldiallylammonium salts. Examples of commercialized products include the products sold under the name “Celquat L 200” and “Celquat H 100” by the company National Starch.

Cationic galactomannan gums such as guar gums containing cationic trialkylammonium groups are described in U.S. Pat. Nos. 3,589,578 and 4,031,307, the disclosures of which are hereby incorporated by reference. Further examples include guar gums modified with a salt (such as chloride) of 2,3-epoxypropyltrimethylammonium. These products are sold, for example, under the trade names JAGUAR C13 S, JAGUAR C 15, JAGUAR C 17 and JAGUAR C162 by the company MEYHALL.

(3) Polymers selected from polymers comprising piperazinyl units, polymers comprising straight and branched, alkylene and hydroxyalkylene divalent radicals, optionally interrupted by aromatic rings, heterocyclic rings, and atoms selected from oxygen, sulphur and nitrogen, and the oxidation and/or quaternization products of these polymers. Such polymers are described in French patents 2,162,025 and 2,280,361, the disclosures of which are hereby incorprated by reference.

(4) Water-soluble polyaminoamides may be prepared, for example, by polycondensation of an acid compound with a polyamine. The polyaminoamides may be crosslinked with an epihalohydrin, a diepoxide, a dianhydride, an unsaturated dianhydride, a diunsaturated derivative, a bishalohydrin, a bisazetidinium, a bishaloacyidiamine, an alkylbishalide, and an oligomer resulting from the reaction of a difunctional compound which with a compound chosen from a bishalohydrin, a bisazetidinium, a bishaloacyidiamine, an alkylbishalide, an epihalohydrin, a diepoxide and a diunsaturated derivative. The crosslinking agent may be employed, for example, in proportions ranging from 0.025 to 0.35 mol per amine group of the polyaminoamide; these polyaminoamides may be alkylated or, if they include one or more tertiary amine functional groups, quaternized. Such polymers are described in French Patents 2,252,840 and 2,368,508, the disclosures of which are hereby incorporated by reference.

(5) Polyaminoamide derivatives resulting from the condensation of polyalkylenepolyamines with polycarboxylic acids, followed by an alkylation with difunctional agents. There may be mentioned, for example, the adipic acid-dialkylaminohydroxyalkyidialkylenetriamine polymers in which the alkyl radical contains from 1 to 4 carbon atoms, such as methyl, ethyl, and propyl. Such polymers are described in French Patent 1,583,363, the disclosure of which is hereby incorporated by reference. Examples of commercial products include the adipic acid/dimethyl-aminohydroxypropyl/diethylenetriamine polymers sold under the name “Cartaretine F, F4 or F8” by the company Sandoz.

(6) Polymers obtained by reaction of a polyalkylenepolyamine containing two primary amine groups and at least one secondary amine group with a dicarboxylic acid chosen from diglycolic acid and saturated aliphatic dicarboxylic acids containing from 3 to 8 carbon atoms. The molar ratio of the polyalkylenepolyamine to the dicarboxylic acid can, for example, range from 0.8:1 to 1.4:1; the polyaminoamide resulting therefrom reacting with epichlorohydrin in a molar ratio of epichlorohydrin relative to the secondary amine group of the polyamino amide of from 0.5:1 to 1.8:1. Such polymers are described in U.S. Pat. Nos. 3,227,615 and 2,961,347, the disclosures of which are hereby incorporated by reference. These polymers are sold, for example, under the name “Hercosett 57” by the company Hercules Inc. and under the name “PD 170” or “Delsette 101” by the company Hercules in the case of the adipic acid/epoxypropyl/diethylenetriamine copolymer.

(7) Cyclopolymers chosen from cyclopolymers of alkyldiallylamine and cyclopolymers of dialkyldiallylammonium, such as the homopolymers and copolymers comprising, as main constituents of the chain, units chosen from formula (XVI) and (XVII):

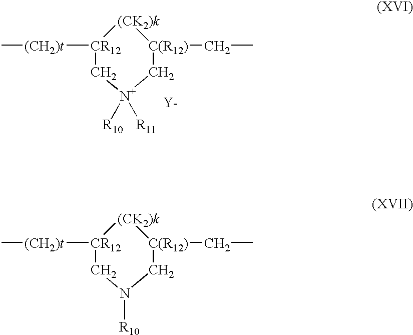

in which formulae

k and t are chosen from the integers 0 and 1, the sum k +t being equal to 1;

R₁₂ is chosen from a hydrogen atom and a methyl radical;

R₁₀ and R₁₁, independently of each other, are chosen from alkyl groups having from 1 to 22 carbon atoms, hydroxyalkyl groups, such as hydroxyalkyl groups in which the alkyl group has 1 to 5 carbon atoms and hydroxyalkyl groups in which the alkyl group has 1 to 4 carbon atoms, and C₁-C₄ amidoalkyl groups. For example, R₁₀ and R₁₁, independently of each other, are chosen from alkyl groups having from 1 to 4 carbon atoms. R₁₀ and R₁₁ can also be chosen from heterocyclic groups which R₁₀ and R₁₁ form together with the nitrogen atom to which they are attached, such as piperidyl and morpholinyl; and

Y⁻¹ is an anion chosen from bromide, chloride, acetate, borate, citrate, tartrate, bisulphate, bisulphite, sulphate and phosphate.

These polymers are described in French patent 2,080,759 and in its Certificate of Addition 2,190,406, the disclosures of which are hereby incorporated by reference.

Examples of commercial products of the polymers defined above include the dimethyldiallylammonium chloride homopolymer sold under the name “Merquat 100” by the company Calgon (and its homologues of low weight-average molecular masses) and the copolymers of diallyl-dimethylammonium chloride and acrylamide sold under the name “MERQUAT 550”.

(8) Quaternary diammonium polymers comprising repeat units chosen from the formula:

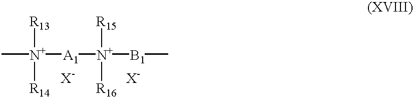

in which:

R₁₃, R₁₄, R₁₅ and R₁₆, which are identical or different, are chosen from aliphatic, alicyclic and arylaliphatic radicals comprising from 1 to 20 carbon atoms, lower hydroxyalkyl aliphatic radicals, heterocyclic rings, and C₁-C₆ alkyl radicals. The heterocyclic rings are formed from R₁₃, R₁₄, R₁₅ and R₁₆, together or separately, with the nitrogen atoms to which they are attached, and the heterocyclic rings optionally contain a second heteroatom other than nitrogen. The C₁-C₆ alkyl radicals are chosen from linear and branched C₁-C₆ alkyl radicals substituted by a group chosen from nitrile groups, ester groups, acyl groups, amide groups and —CO—O—R₁₇-D and —CO—NH—R₁₇-D groups where R₁₇ is an alkylene and D is a quaternary ammonium group;

A₁ and B₁ are chosen from linear and branched, saturated and unsaturated polymethylene groups comprising from 2 to 20 carbon atoms. The polymethylene groups may contain, bonded to or inserted into the main chain, at least one entity chosen from aromatic rings, oxygen and sulphur atoms, sulphoxide, sulphone, disulphide, aminos, alkylaminos, hydroxyl, quaternary ammoniums, ureidos, amides and esters;

X⁻ is an anion chosen from anions derived from an inorganic and organic acids, such as chloride and bromide;

A₁, R₁₃ and R₁₅, with the two nitrogen atoms to which they are attached, may form a piperazine ring. Additionally, if A₁ is chosen from saturated and unsaturated, linear and branched alkylene and hydroxyalkylene radicals, B₁ may also denote a group (CH₂)ₙ—CO—D—OC—(CH₂)ₙ—

in which D is chosen from:

a) a glycol residue of formula: —O—Z—O—, where Z is chosen from linear and branched hydrocarbon radicals and groups corresponding to one of the following formulae:

—(CH₂—CH₂—O)ₓ—CH₂—CH₂—

—[CH₂—CH(CH₃)—O]_{y}—CH₂—CH(CH₃)—

where x and y are chosen from an integer from 1 to 4, representing a defined and unique degree of polymerization and any number from 1 to 4 representing a mean degree of polymerization;

b) a disecondary diamine residue such as a piperazine derivative;

c) a diprimary diamine residue of formula: —NH—Y—NH—, where Y is chosen from linear and branched hydrocarbon radicals and the divalent radical

—CH₂—CH₂—S—S—CH₂—CH₂—;

d) a ureylene group of formula: —NH—CO—NH—.

These polymers have a number-average molecular mass which is generally from 1000 to 100,000.

Polymers of this type are described in French Patents 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020, the disclosures of which are hereby incorporated by reference.

Use may also be made of polymers which comprise repeat units corresponding to the formula:

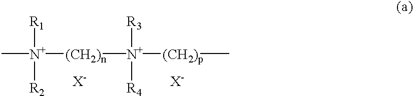

in which R₁, R₂, R₃ and R₄, which are identical or different, are chosen from alkyl and hydroxyalkyl radicals having from 1 to 4 carbon atoms approximately;

n and p are chosen from integers from 2 to 20 approximately; and

X⁻ is an anion chosen from anions derived from inorganic and organic acids

An example of a compound of formula (a) is that for which R₁, R₂, R₃ and R₄ represent a methyl radical and n=3, p=6 and X=Cl, called Hexadimethrine chloride according to the INCI nomenclature (CTFA).

(9) Quaternary polyammonium polymers comprising units of formula (XIX):

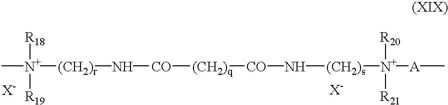

in which:

R₁₈, R₁₉, R₂₀ and R₂₁, which are identical or different, are chosen from a hydrogen atom and methyl, ethyl, propyl, β-hydroxyethyl, β-hydroxypropyl and —CH₂CH₂(OCH₂CH₂)ₚOH radicals, where p is chosen from 0 or an integer from 1 to 6, provided that R₁₈, R₁₉, R₂₀ and R₂₁ do not simultaneously represent a hydrogen atom;

r and s, which are identical or different, are chosen from integers from 1 to 6;

q is chosen from 0 and an integer from 1 to 34;

X is an anion such as a halide; and

A is chosen from radicals of dihalides and —CH₂—CH₂—O—CH₂—CH₂—.

These compounds are described in Patent Application EP—A-122 324, the disclosure of which is hereby incorporated by reference. Examples of commercial products of these compounds include the products sold under the names “Mirapoly® A 15”, “Mirapol® AD1 ”, “Mirapol® AZ1” and “Mirapol® 175” by the company Miranol.

(10) Quaternary vinylpyrrolidone and vinylimidazole polymers such as, for example, the products marketed under the names Luviquat® FC 905, FC 550 and FC 370 by the company B.A.S.F.

(11) Polyamines such as the product sold under the name Polyquart® H by HENKEL, referred to under the name of “POLYETHYLENE GLYCOL (15) TALLOW POLYAMINE” in the CTFA dictionary.

(12) The crosslinked polymers of methacryloyloxy(C₁-C₄ alkyl)tri(C₁-C₄ alkyl)ammonium salts such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, such as methylenebisacrylamide. As an example, a crosslinked acrylamide/ methacryloyloxyethyltrimethylammonium chloride copolymer (20/80 by weight) may be employed in the form of a dispersion containing 50% by weight of the said copolymer in mineral oil. This dispersion is sold under the name of “SALCARE® SC 92” by the company ALLIED COLLOIDS. As another example, a crosslinked methacryloyloxyethyltrimethylammonium chloride homopolymer may be employed containing approximately 50% by weight of the homopolymer in mineral oil or in a liquid ester. These dispersions are sold under the names of “SALCARE® SC 95” and “SALCARE® SC 96” by the company ALLIED COLLOIDS.

Other cationic polymers that may be employed within the scope of the invention are chosen from cationic proteins and hydrolysates of cationic proteins, polyalkyleneimines,such as polyethyleneimines, polymers containing units chosen from vinylpyridine and vinylpyridinium units, condensates of polyamines and of epichlorohydrin, quaternary polyureylenes and chitin derivatives.

Among the cationic polymers capable of being used within the scope of the present invention, reference may be made to quaternary cellulose ether derivatives such as the products sold under the name “JR 400” by the company UNION CARBIDE CORPORATION, cationic cyclopolymers, such as the homopolymers or copolymers of dimethyldiallylammonium chloride, sold under the names “MERQUAT 100”, “MERQUAT 550” and “MERQUAT S” by the company CALGON, the quaternary polymers of vinylpyrrolidone and of vinylimidazole and mixtures thereof.

The cationic proteins and protein hydrolysates are, for example, chemically modified polypeptides carrying quaternary ammonium groups at the chain end, or grafted on the latter. Their molecular mass may vary, for example, from 1 500 to 10 000, such as from 2 000 to 5 000 approximately. Among these compounds, examples include:

collagen hydrolysates carrying triethylammonium groups such as the products sold under the name “Quat-Pro E” by the company MAYBROOK and called in the CTFA dictionary “Triethonium Hydrolyzed Collagen Ethosulfate”;

collagen hydrolysates carrying trimethylammonium and trimethylstearylammonium chloride groups, sold under the name “Quat-Pro S” by the company MAYBROOK and called in the CTFA dictionary “Steartrimonium Hydrolyzed Collagen”;

animal protein hydrolysates carrying trimethylbenzylammonium groups such as the products sold under the name “Crotein BTA” by the company CRODA and called in the CTFA dictionary “Benzyltrimonium hydrolyzed animal protein”;

protein hydrolysates carrying, on the polypeptide chain, quaternary ammonium groups comprising at least one alkyl radical having from 1 to 18 carbon atoms.

Among these protein hydrolysates, there may be mentioned, inter alia:

“Croquat L” in which the quaternary ammonium groups comprise a C₁₂ alkyl group;

“Croquat M” in which the quaternary ammonium groups comprise C₁₀-C₁₈ alkyl groups;

“Croquat S” in which the quaternary ammonium groups comprise a C₈ alkyl group;

“Crotein Q” in which the quaternary ammonium groups comprise at least one alkyl group having from 1 to 18 carbon atoms.

These various products are sold by the company Croda.

Other quaternized proteins or hydrolysates are, for example, those corresponding to the formula:

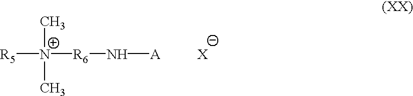

in which X⁻ is chosen from an anion of an organic acid and an anion of an inorganic acid;

A is chosen from protein residues derived from protein hydrolysates, such as collagen;

R₅ is chosen from a lipophilic groups comprising up to 30 carbon atoms;

R₆ is an alkylene group having 1 to 6 carbon atoms.

There may also be mentioned, for example, the products sold by the company Inolex, under the name “Lexein QX 3000”, called in the CTFA dictionary “Cocotrimonium Collagent Hydrolysate”.

There may also be mentioned quaternized plant proteins such as wheat, maize or soyabean proteins: as quaternized wheat proteins. Additional examples include those products sold by the company Croda under the names “Hydrotriticum WQ or QM”, called in the CTFA dictionary “Cocodimonium Hydrolysed wheat protein”, “Hydrotriticum QL” called in the CTFA dictionary “Laurdimonium hydrolysed wheat protein” and “Hydrotriticum QS”, called in the CTFA dictionary “Steardimonium hydrolysed wheat protein”.

The quaternary ammonium salts comprising at least one amide functional group may be chosen from those which have the following formula (XXI):

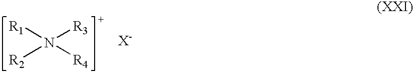

in which the radicals R₁ to R₄, which may be identical or different, are chosen from the radicals: alkyl having from 1 to 30 carbon atoms, (C₂-C₆)polyoxyalkylene, (C₁₂-C₂₂)alkylamido(C₂-C₆)alkyl, (C₁₂-C₂₂)alkyl acetate, and hydroxyalkyl comprising about from 1 to 30 carbon atoms, provided, however, that at least one of the radicals contains at least one amide functional group; and

X⁻ is an anion chosen from halides, phosphates, acetates, lactates, (C₂-C₆)alkyl sulphates, and alkyl and alkylaryl sulphonates,

Examples among the quaternary ammonium salts of formula (XXI) include palmitylamidopropyltrimethylammonium chloride and stearamidopropyldimethyl(myristyl acetate)ammonium chloride sold under the name CERAPHYL® 70 by the company VAN DYK.

The quaternary ammonium salts of imidazolinium are chosen from those of the following formula (XXII):

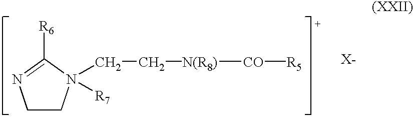

in which

R₅ is chosen from alkenyl and alkyl radicals comprising from 8 to 30 carbon atoms, such as alkenyl and alkyl radicals derived from tallow fafty acids;

R₆ is chosen from a hydrogen atom, C₁-C₄ alkyl radicals and alkenyl and alkyl radicals comprising from 8 to 30 carbon atoms;

R₇ is chosen from C₁-C₄ alkyl radicals;

R₈ is chosen from a hydrogen atom and C₁-C₄ alkyl radicals; and

X is an anion chosen from halides, phosphates, acetates, lactates, alkyi sulphates, and alkyl and alkylaryl sulphonates.

Examples of the quaternary ammonium salts of imidazolinium are compounds corresponding to the formula above wherein R₅ and R₆ are chosen from a mixture of alkenyl and alkyl radicals comprising from 12 to 21 carbon atoms, such as alkenyl and alkyl radicals derived from tallow fatty acids, R₇ is methyl, and R₈ is hydrogen. Such a product is, for example, marketed under the name “REWOQUAT W 75” by the company REWO.

The quaternary diammonium salts are chosen from those of formula (XXIII):

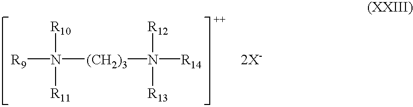

in which R₉ is chosen from aliphatic radicals comprising from about 16 to 30 carbon atoms;

R₁₀, R₁₁, R₁₂, R₁₃ and R₁₄, which are identical or different, are chosen from hydrogen and alkyl radicals comprising from 1 to 4 carbon atoms; and

X⁻ is an anion chosen from halides, acetates, phosphates, nitrates and methyl sulphates. An example of a quaternary diammonium salt of formula (XXIII) is propanetallowdiammonium dichloride.

The quaternary ammonium salts containing at least one ester functional group which can be used according to the invention are, for example, those of the following formula (XXIV):

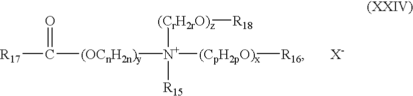

in which:

R₁₅ is chosen from linear and branched C₁-C₆ alkyl, C₁-C₆ hydroxyalkyl, and C₁-C₆dihydroxyalkyl radicals;

R₁₆ is chosen from:

a hydrogen atom, the radical

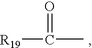

saturated and unsaturated, linear and branched C₁-C₂₂ hydrocarbon radicals R₂₀,

R₁₈ is chosen from:

a hydrogen atom, the radical

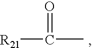

saturated and unsaturated, linear and branched C₁-C₆ hydrocarbon radicals R₂₂,

R₁₇, R₁₉ and R₂₁, which are identical or different, are chosen from saturated and unsaturated, linear and branched C₇-C₂₁ hydrocarbon radicals;

n, p and r, which are identical or different, are chosen from integers ranging from 2 to 6;

y is chosen from an integer ranging from 1 to 10;

x and z, which are identical or different, are chosen from integers ranging from 0 to 10; and

X⁻ is chosen from simple and complex, organic and inorganic anions;

provided, however, that the sum x+y+z is equal to 1 to 15, that when x is equal to 0, then R₁₆ is R₂₀ and that when z is equal to 0, then R₁₈ is R₂₂.

For example, R₁₅ can be linear. Further examples of the quaternary diammonium salts are compounds chosen from the formula above wherein R₁₅ is chosen from methyl, ethyl, hydroxyethyl and dihydroxypropyl radicals, R₁₆ is chosen from hydrocarbon radicals R₂₀ having from 12 to 22 carbon atoms and hydrocarbon radicals having from 1 to 3 carbon atoms, R₁₈ is chosen from hydrocarbon radicals R₂₂ having from 1 to 3 carbon atoms, R₁₇, R₁₉ and R₂₁, are chosen from saturated and unsaturated, linear and branched C₁₁-C₂₁ hydrocarbon radicals, such as saturated and unsaturated, linear and branched C₁₁-C₂₁ alkyl and alkenyl radicals, x and z are integers chosen from 0 and 1, y is 1, n, p and r are equal to integers chosen from 2 and 3, such as 2, and/or X⁻ is chosen from halides such as chloride, bromide, and iodide and alkyl sulphates, such as methyl sulphate, methane sulphonate, phosphate, nitrate, tosylate, anions derived from an organic acid such as acetate or lactate, and ammonium compatible anions containing an ester functional group.

A further example of a quaternary diammonium salt is a compound corresponding to the formula above wherein X⁻ is chosen from chloride and methyl sulphate, and the sum of x +y +z equals an integer from 1 to 10.

Additional examples of ammonium salts are those of formula (XXIV) in which:

R₁₅ is chosen from methyl and ethyl radicals;

x and y are equal to 1;

z is an integer chosen from 0 and 1;

n, p and r are equal to 2;

R₁₆ is chosen from:

the radical

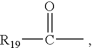

methyl, ethyl and C₁₄-C₂₂ hydrocarbon radicals and a hydrogen atom;

R₁₈ is chosen from:

the radical

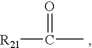

and a hydrogen atom; and

R₁₇, R₁₉ and R₂₁, which may be identical or different, are chosen from saturated and unsaturated, linear and branched C₁₃-C₁₇ hydrocarbon radicals, such as saturated and unsaturated, linear and branched C₁₃-C₁₇ alkyl and alkenyl radicals and further such as saturated and unsaturated, linear C₁₃-C₁₇ alkyl and alkenyl radicals.

There may be mentioned, for example, compounds of formula (XXIV) such as the salts, such as the chloride and methyl sulfate salts, of diacyloxyethyldimethylammonium, diacyloxyethylhydroxyethylmethylammonium, monoacyloxyethyldihydroxyethylmethylammonium, triacyloxyethylmethylammonium, monoacyloxyethylhydroxyethyldimethylammonium and mixtures thereof. The acyl radicals, for example, have 14 to 18 carbon atoms and may be obtained, for example, from a vegetable oil, such as palm and sunflower oil. When the compound contains several acyl radicals, the latter may be identical or different.

These products are obtained, for example, by direct esterification of optionally oxyalkylenated triethanolamine, triisopropanolamine, alkyldiethanolamine or alkyldiisopropanolamine with fatty acids or with mixtures of fatty acids of plant or animal origin or by transesterification of their methyl esters. This esterification is followed by quaternization using an alkylating agent such as an alkyl (such as methyl and ethyl) halide, a dialkyl (such as methyl and ethyl) sulphate, methyl methanesulphonate, methyl para-toluenesulphonate, glycol and glycerol chlorohydrin.

Such compounds are, for example, marketed under the names DEHYQUART by the company HENKEL, STEPANQUAT by the company STEPAN, NOXAMIUM by the company CECA, and REWOQUAT WE 18 by the company REWO-WITCO.

Thus, for example, the composition of the invention may contain a mixture of quaternary ammonium mono-, di- and triester salts with a majority, by weight, of diester salts.

Examples of the mixture of ammonium salts include the mixture containing 15 to 30% by weight of acyloxyethyldihydroxyethylmethylammonium methyl sulphate, 45 to 60% of diacyloxyethylhydroxyethylmethylammonium methyl sulphate and 15 to 30% of triacyloxyethylmethyl-ammonium methyl sulphate, the acyl radicals having from 14 to 18 carbon atoms and being obtained from optionally partially hydrogenated palm oil.

The ammonium salts containing at least one ester functional group which are disclosed in Patents U.S. Pat. No. 4,874,554 and U.S. Pat. No. 4,137,180, the disclosures of which are hereby incorporated by reference, may also be used.

According to the invention, the soluble conditioning agent(s) according to the invention may, for example, represent from 0.001% to 20% by weight, for example, from 0.01% to 10% by weight, such as from 0.05 to 3% by weight, relative to the total weight of the final composition.

The compositions of the invention may also contain, in addition, at least one other surfactant which is, for example, present in a quantity of from 0.1% to 40% by weight approximately, for example from 3% to 30%, such as from 5% to 20%, relative to the total weight of the composition.

This surfactant may be chosen from anionic, amphoteric and nonionic surfactants, and mixtures thereof.

Additional surfactants for carrying out the present invention can be chosen from the following:

(i) Anionic Surfactant(s):

Their nature may not be of critical importance within the context of the present invention.

Thus, by way of example of anionic surfactants that can be employed, by themselves or as mixtures, in the context of the present invention, there may be mentioned (nonlimiting list) the salts (for example alkali metal salts such as sodium, ammonium salts, amine salts, amino alcohol salts and magnesium salts) of the following compounds: alkyl sulphates, alkyl ether sulphates, alkylamido ether sulphates, alkylaryl-polyether sulphates, monoglyceride sulphates, alkyl sulphonates, alkyl phosphates, alkylamidesulphonates, alkyl aryl sulphonates, α-olefinsulphonates, paraffin-sulphonates, alkyl sulphosuccinates, alkyl ether sulphosuccinates, alkylamidesulphosuccinates, alkyl sulphosuccinamates, alkyl sulphoacetates, alkyl ether phosphates, acyl sarcosinates, acyl isethionates and N-acyltaurates, the alkyl or acyl radical of all these different compounds containing from 8 to 24 carbon atoms, and the aryl radical chosen from a phenyl and benzyl group. Among the anionic surfactants there may also be mentioned the salts of fatty acids such as the salts of oleic, ricinoleic, palmitic and stearic acids, the acids of copra oil or of hydrogenated copra oil, and acyl lactylates in which the acyl radical contains 8 to 20 carbon atoms. Weakly anionic surfactants may also be employed such as alkyl-D-galactosideuronic acids and salts thereof, as well as the polyoxyalkylenated carboxylic (C₆-C₂₄)alkyl ether acids, the polyoxyalkylenated carboxylic (C₆-C₂₄)alkylaryl ether acids, the polyoxyalkylenated carboxylic (C₆-C₂₄)alkyl amidoether acids and their salts, such as those containing from 2 to 50 ethylene oxide groups and mixtures thereof.

Additional examples of anionic surfactants include the salts of alkyl sulphates and of alkyl ether sulphates and mixtures thereof.

(ii) Nonionic Surfactant(s):

The nonionic surfactants themselves are also compounds which are well known per se (in this respect see the “Handbook of Surfactants” by M. R. Porter, published by Blackie & Son (Glasgow and London), 1991, pp. 116-178) and, in the context of the present invention, their nature may not be of critical importance. They can thus be chosen from (nonlimiting list) alcohols, alpha-diols, alkylphenols and polyethoxylated, polypropoxylated or polyglycerolated fatty acids which have a fatty chain containing, for example, 8 to 18 carbon atoms, it being possible, for example, for the number of ethylene oxide or propylene oxide groups to range from 2 to 50 and it being possible, for example, for the number of glycerol groups to range from 2 to 30. The copolymers of ethylene oxide and propylene oxide and the condensates of ethylene oxide and propylene oxide with fatty alcohols may also be mentioned by way of example. Examples of these copolymers and condensates include the polyethoxylated fatty amides containing, for example, from 2 to 30 mol of ethylene oxide, the polyglycerolated fatty amides on average containing, for example, 1 to 5 glycerol groups, such as 1.5 to 4, the polyethoxylated fatty amines containing, for example, 2 to 30 mol of ethylene oxide, the oxyethylenated fatty acid esters of sorbitan containing, for example, from 2 to 30 mol of ethylene oxide, the fatty acid esters of sucrose, the fatty acid esters of polyethylene glycol, alkylpolyglycosides, N-alkylglucamine derivatives, amine oxides such as the oxides of (C₁₀-C₁₄) alkylamines, and the N-acylaminopropylmorpholine oxides. Alkylpolyglycosides are still another example of nonionic surfactants for the present invention.

(iii) Amphoteric Surfactant(s):

The additional amphoteric surfactants, the nature of which may not be of critical importance in the context of the present invention, may be (nonlimiting list) chosen from derivatives of aliphatic secondary and tertiary amines in which the aliphatic radical is chosen from linear and branched chains comprising 8 to 22 carbon atoms and comprising at least one water-solubilizing anionic group (for example carboxylate, sulphonate, sulphate, phosphate or phosphonate); (C₈-C₂₀)alkylbetaines, sulphobetaines, (C₈-C₂₀)alkylamido(C₁-C₆)alkylbetaines and (C₈-C₂₀)alkylamido(C₁-C₆)al kylsulphobetaines.

Among the amine derivatives there may be mentioned the products sold under the name Miranol, as described in U.S. Pat. No. 2,528,378 and U.S. Pat. No. 2,781,354, the disclosures of which are hereby incorporated by reference and of structures:

R₂—CONHCH₂CH₂—N(R₃)(R₄)(CH₂COO—) (2)

in which: R₂ is chosen from alkyl radicals derived from an acid R₂—COOH present in hydrolysed copra oil, such as a heptyl, nonyl and undecyl radicals;

R₃ is a beta-hydroxyethyl group; and R₄ is chosen from a carboxymethyl group and

R₅—CONHCH₂CH₂—N(B)(C) (3)

in which:

B is chosen from —CH₂CH₂OX′;

(C) represents —(CH₂)₂—Y″, wherein z is an integer chosen from 1 and 2;

X′ is chosen from the radical —CH₂CH₂—COOH and a hydrogen atom;

Y′ is chosen from —COOH and the radical —CH2—CHOH—SO3H; and

R₅ is chosen from alkyl radicals of a carboxylic acid present in an oil chosen from copra oil and hydrolysed linseed oil, such as C₇, C₉, C₁₁, C₁₃, and C₁₇ alkyl radicals, iso C₁₇ alkyl radicals and unsaturated C₁₇ radicals.

These compounds are classified in the CTFA dictionary, 7th edition, 1997, under the names Disodium Cocoamphodiacetate, Disodium Lauroamphodiacetate, Disodium Capryloamphodiacetate, Disodium Caproamphodiacetate, Disodium Cocoamphodipropionate, Disodium Lauroamphodipropionate, Disodium Caproampho-dipropionate, Disodium Capryloamphodipropionate, Lauro-amphodipropionic acid, and Cocoamphodipropionic acid.

By way of example, there may be mentioned the commercial product of disodium cocoamphodiacetate marketed under the trade name MIRANOL® C2M concentrate by the company RHODIA CHIMIE.

Thus, representative compositions of the invention may include mixtures of surfactants such as mixtures of anionic surfactants and mixtures of anionic surfactants and amphoteric and/or nonionic surfactants.

Examples of such mixtgures of surfactants include the following anionic surfactants: sodium, triethanolamine and ammonium (C₁₂-C₁₄)alkyl sulphates, oxyethylenated sodium, triethanolamine and ammonium (C₁₂-C₁₄)alkyl ether sulphates containing 2.2 mol of ethylene oxide, sodium cocoyl isethionate and sodium (C₁₄-C₁₆)alphaolefin sulphonate mixed with an amphoteric surfactant.

The amphoteric surfactant in such representative mixtures may, for example, be chosen from the amine derivatives called disodium cocoamphodipropionate and sodium cocoamphopropionate marketed by the company RHODIA CHIMIE under the trade name “MIRANOL® C2M CONC” in aqueous solution at 38% of active substance or under the name MIRANOL® C32 and alkylbetaines, such as cocobetaine marketed under the name “DEHYTON® AB 30” in aqueous solution at 32% AS by the company HENKEL and the (C₈-C₂₀)alkylamido(C₁-C₆)alkylbetaines, such as TEGOBETAINE® F 50 marketed by the company GOLDSCHMIDT.

Anionic surfactant(s) different from the N-acylated compounds of mono- and polyamidated polycarboxylic amino acids according to the invention are generally, for example, present in an amount of about 1 to about 30% by weight such as 3 to 15% by weight, relative to the total weight of the composition.

The amphoteric or nonionic surfactant(s) are generally present, for example, in an amount of about 0.5 to about 15% by weight such as from 1 to 5% by weight, relative to the total weight of the composition.

The quantity and the quality of the surfactants are those sufficient to confer a satisfactory foaming and/or detergent power on the final composition.

In the composition according to the present invention, all the detergent surfactants generally, for example, represent from 4 to 50% by weight, for further example, from 6 to 35% by weight such as from 8 to 25% by weight relative to the total weight of the composition.

The composition of the invention may also contain at least one additive chosen from thickeners, perfumes, pearlescent agents, preservatives, sunscreens, silicones, ceramides, pseudoceramides, linear and branched chain C₁₆-C₄₀ acids such as 18-methyleicosanoic acid, hydroxy acids, vitamins, provitamins such as panthenol, vegetable oils, mineral oils and synthetic oils, antidandruff agents and any other additive conventionally used in the cosmetic field which does not affect the stability and the properties of the compositions according to the invention.

These additives may be present in the composition according to the invention in proportions which may, for example, range from 0.0001 to 50% by weight relative to the total weight of the composition. The precise quantity of each additive is easily determined by a person skilled in the art according to its nature and its function.

The cosmetically acceptable medium may consist solely of water or the cosmetically acceptable medium may comprise a mixture of water and a cosmetically acceptable solvent, for example, a lower C₁-C₄ alcohol, such as ethanol, isopropanol, tert-butanol and n-butanol, and alkylene glycols, such as propylene glycol and glycol ethers. By way of example, the composition may comprise from 50 to 95% by weight of water relative to the total weight of the composition.

The detergent compositions according to the invention have a final pH which is generally, for example, from 3 to 10, such as a pH from 4 to 8. The adjustment of the pH to the desired value may be carried out conventionally by addition of a base (organic or inorganic) to the composition, for example aqueous ammonia and primary, secondary and tertiary (poly)amines, such as monoethanolamine, diethanolamine, triethanolamine, isopropanolamine and 1,3-propanediamine, or by the addition of an acid, for example a carboxylic acid such as citric acid.

The compositions in accordance with the invention may comprise, in addition to the combination defined above, viscosity-regulating agents such as electrolytes or thickening agents. As examples there may be mentioned sodium chloride, scleroglucans, xanthan gums, fatty acid alkanolamides, alkyl ether carboxylic acid alkanolamides which are optionally oxyethylenated with up to 5 mol of ethylene oxide, such as the product marketed under the name “AMINOL A15” by the company CHEM Y, crosslinked polyacrylic acids and crosslinked acrylic acid/C₁₀-C₃₀ alkyl acrylate copolymers. These viscosity-regulating agents are used in the compositions according to the invention in proportions which may be up to 10% by weight relative to the total weight of the composition.

The compositions in accordance with the invention may also contain up to 5% of pearlescent or opacifying agents well known in the state of the art, such as sodium and magnesium palmitates, sodium and magnesium stearates and hydroxystearates, fatty chain-containing acylated derivatives, such as ethylene glycol and polyethylene glycol monostearates and distearates, and fatty chain-containing ethers such as distearyl ether and 1-(hexadecyloxy)-2-octadecanol.

The compositions according to the invention may also contain foam synergists selected from C₁₀-C₁₈ 1,2-alkanediols and fatty alkanolamides derived from mono- and diethanolamine.

The compositions in accordance with the invention may be used for washing and treating a keratinous material such as the hair, the skin, the eyelashes, the eyebrows, the nails, the lips, the scalp. A common example would be treatement of hair.

Examples of the detergent compositions according to the invention are shampoos, shower gels and foam baths. The compositions of the invention may also be provided in the form of rinse-off after-shampoos and leave-in after-shampoos, of compositions for permanent waving, straightening, dyeing or bleaching, or in the form of rinse-off compositions, to be applied before or after dyeing, bleaching, permanent waving or straightening or between the two stages of permanent waving or straightening. The compositions of the invention may also be provided in the form of make-up removing products.

The compositions according to the invention may be provided in forms selected from gels, milks, creams, emulsions, thickened lotions thickened foams and may be used for the skin, the scalp, the nails, the eyelashes, the lips and the hair.

These detergent compositions may be foaming and the foaming power of the compositions according to the invention, characterized by a foam height, is generally greater than 75 mm, such as greater than 100 mm, measured according to the modified ROSS-MILES method (NF T 73-404/ISO696). The modified method is as follows: The measurement is carried out at the temperature of 22° C. with osmosed water. The concentration of the solution is 2 g/l. The drop height is 1 m. The quantity of composition which drops is 200 ml. These 200 ml of composition fall into a measuring cylinder having a diameter of 50 mm and containing 50 ml of the composition tested. The measuring is made 5 minutes after stopping the flow of the composition.

The subject of the invention is also a method for treating a keratinous material, such as the skin and the hair, characterized in that it comprises applying to the keratinous material a cosmetic composition as defined above, and then optionally rinsing off, with water, for example.

Thus, the method according to the invention allows the treatment of, care of, washing of, and/or removal of make-up from the skin, the hair or any other keratinous material.

In the text which follows or in the preceding text, the percentages expressed are by weight. The invention will now be illustrated more fully with the aid of the following examples which cannot be considered as limiting the embodiments described. In the examples, AS means active substance.

### EXAMPLE 1

Three shampoo compositions were prepared, one in accordance with the invention (composition A) and the other 2 comparatives (compositions B and C):

| | A | | |
|---|---|---|---|
| | (invention) | B | C |
| Triethanolamine salt of | 16.7 g | — | — |
| N-cocoylglutamine at 30% | (5 gAS) | | |
| AS (Foam up doucé GM from | | | |
| KYOWA HAKKO) | | | |
| Cocoylbetaine as an aqueous | 8.3 g | 8.3 g | 8.3 g |
| solution at 30% AS | (2.5 gAS) | (2.5 gAS) | (2.5 gAS) |
| (DEHYTON AB 30 from | | | |
| COGNIS) | | | |
| Sodium (C₁₂-C₁₄)alkyl | 38 g | 38 g | 57 g |
| ether sulphate oxyethylenated | (9.9 gAS) | (9.9 gAS) | (14.8 gAS) |
| with 2.2 mol of ethylene | | | |
| oxide as an aqueous solution | | | |
| at 26% of AS | | | |
| N-cocoyl glutamate as an | — | 5 gAS | — |
| aqueous solution at 30% AS | | | |
| (Acyl glutamate CT12 | | | |
| from AJINOMOTO) | | | |
| Hydroxyethylcellulose | 1 g | 1 g | 1 g |
| quaternized with 2,3- | | | |
| epoxypropyltrimethyl- | | | |
| ammonium (JR400 from | | | |
| AMERCHOL) | | | |
| Preservatives | qs | qs | qs |
| pH agent qs pH | 6.5 | 6.5 | 6.5 |
| Demineralized water qs | 100 g | 100 g | 100 g |

Shampooing is performed by applying about 1 g of composition A to locks of 2.5 g of previously wet sensitized hair. The shampoo is caused to lather, an exposure time of 10 minutes allowed to elapse and then abundantly rinsed off with water. The locks are dried for 10 minutes at 60° C. A second application of the composition is carried out.

The same procedure as above is used with the comparative compositions B and C.

A panel of experts evaluated the appearance of the wet hair.

100% of the experts indicated that the hair treated with composition A according to the invention was significantly sleeker and softer than that treated with compositions B and C.

### EXAMPLE 2

Two shampoo compositions were prepared, one in accordance with the invention (composition D) and the other comparative (composition E):

| | D | |
|---|---|---|
| | (invention) | E |
| Triethanolamine salt of N-cocoyl | 16.7 g | — |
| glutamine at 30% AS (Foam up | (5 gAS) | |
| doucé GM from KYOWA HAKKO) | | |
| Cocoylbetaine as an aqueous | 8.3 g | 8.3 g |
| solution at 30% AS (DEHYTON AB | (2.5 gAS) | (2.5 gAS) |
| 30 from COGNIS) | | |
| Sodium (C₁₂-C₁₄)alkyl | 38 g | 57 g |
| ether sulphate oxyethylenated with | (9.9 gAS) | (14.8 gAS) |
| 2.2 mol of ethylene oxide as an | | |
| aqueous solution at 26% of AS | | |
| Hydroxyethylcellulose grafted | 1 g | 1 g |
| with a methacryloylethyl | | |
| methacryloylethyl | | |
| trimethylammonium salt | | |
| (CELQUAT from NATIONAL STARCH) | | |
| Preservatives | qs | qs |
| pH agent qs pH | 6.5 | 6.5 |
| Demineralized water qs | 100 g | 100 g |

Shampooing is performed by applying about 1 g of composition D to locks of 2.5 g of previously wet sensitized hair. The shampoo is caused to lather, an exposure time of 10 minutes allowed to elapse and then abundantly rinsed off with water. The locks are dried for 10 minutes at 60° C. A second application of the composition is carried out.

The same procedure as above is used with the comparative composition E.

A panel of experts evaluated the appearance of the wet hair.

100% of the experts indicated that the hair treated with composition D according to the invention was significantly sleeker and disentangled more easily than that treated with composition E.

### EXAMPLE 3

Two shampoo compositions were prepared, one in accordance with the invention (composition F) and the other comparative (composition G):

| | F | |
|---|---|---|
| | (invention) | G |
| Triethanolamine salt of N-cocoyl | 16.7 g | — |
| glutamine at 30% AS (Foam up | (5 gAS) | |
| doucé GM from KYOWA HAKKO) | | |
| Cocoylbetaine as an aqueous | 8.3 g | 8.3 g |
| solution at 30% AS (DEHYTON AB | (2.5 gAS) | (2.5 gAS) |
| 30 from COGNIS) | | |
| Sodium (C₁₂-C₁₄)alkyl | 38 g | 57 g |
| ether sulphate oxyethylenated with | (9.9 gAS) | (14.8 gAS) |
| 2.2 mol of ethylene oxide as an | | |
| aqueous solution at 26% of AS | | |
| Polyurethane-1 (LUVISET PUR | 8.3 g | 8.3 g |
| from BASF) | (2.5 gAS) | (2.5 gAS) |
| Preservatives | qs | qs |
| pH agent qs pH | 6.5 | 6.5 |
| Demineralized water qs | 100 g | 100 g |

Shampooing is performed by applying about 1 g of composition F to locks of 2.5 g of previously wet sensitized hair. The shampoo is caused to lather, an exposure time of 10 minutes allowed to elapse and then abundantly rinsed off with water. The locks are dried for 10 minutes at 60° C. A second application of the composition is carried out.

The same procedure as above is used with the comparative composition G.

A panel of experts evaluated the appearance of the wet hair.

90% of the experts indicated that the hair treated with composition F according to the invention was significantly sleeker and/or softer and/or disentangled more easily than that treated with composition G.

### EXAMPLE 4

Two shampoo compositions were prepared, one in accordance with the invention (composition H) and the other comparative (composition I):

| | H | |
|---|---|---|
| | (invention) | I |
| Triethanolamine salt of N-cocoyl | 16.7 g | — |
| glutamine at 30% AS (Foam up | (5.01 gAS) | |
| doucé GM from KYOWA HAKKO) | | |
| Cocoylbetaine as an aqueous | 8.3 g | 8.3 g |
| solution at 30% AS (DEHYTON AB | (2.49 gAS) | (2.49 gAS) |
| 30 from COGNIS) | | |
| Sodium (C₁₂-C₁₄)alkyl | 38 g | 57 g |
| ether sulphate oxyethylenated with | (9.88 gAS) | (14.82 gAS) |
| 2.2 mol of ethylene oxide as an | | |
| aqueous solution at 26% of AS | | |
| Copolymer of methacryloylethyl | 1 g | 1 g |
| N,N-dimethyl carboxymethyl betaine | (0.22 gAS) | (0.22 gAS) |
| and methyl methacrylate as am | | |
| aqueous solution at 22% of AS | | |
| Preservatives | qs | qs |
| pH agent qs pH | 6.5 | 6.5 |
| Demineralized water qs | 100 g | 100 g |

Shampooing is performed by applying about 1 g of composition H to locks of 2.5 g of previously wet sensitized hair. The shampoo is caused to lather, an exposure time of 10 minutes allowed to elapse and then abundantly rinsed off with water. The locks are dried for 10 minutes at 60° C. A second application of the composition is carried out.

The same procedure as above is used with the comparative composition I.

A panel of experts evaluated the appearance of the wet hair.

100% of the experts indicated that the hair treated with composition H according to the invention was significantly sleeker and/or softer than that treated with composition I.

We claim:

1. A detergent cosmetic composition comprising, in a cosmetically acceptable medium, (a) at least one anionic surfactant chosen from N-acylated compounds of mono- and polyamidated polycarboxylic amino acids and the salts of said acids and (b) at least one soluble conditioning agent chosen from:
anionic polymers,
nonionic polymers,
cationic polymers,
amphoteric polymers,
hydrolysates of cationic polymers, and
cationic surfactants chosen from quaternary ammonium
salts comprising at least one amide functional group, quaternary ammonium salts of imidazoline, quaternary diammonium salts, quaternary ammonium salts comprising at least one ester functional group.

2. The composition of claim 1 wherein said at least one anionic surfactant is chosen from N-acylated compounds of mono- and polyamidated polycarboxylic amino acids of the following formula (I): 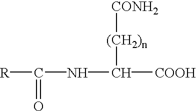
wherein:
R is chosen from linear and branched, saturated and unsaturated hydrocarbon radicals comprising from 5 to 29 carbon atoms, and
n is an integer chosen from 1 and 2.

3. The composition of claim 2 wherein R is chosen from mono- and polyunsaturated alkyl and alkenyl radicals comprising from 5 to 29 carbon atoms.

4. The composition of claim 3 wherein the alkyl and alkenyl radicals comprise from 7 to 22 carbon atoms.

5. The composition of claim 1 wherein said at least one anionic surfactant is an N-cocoylglutamine salt.

6. The composition of claim 1 wherein the anionic polymers are chosen from:
polymers comprising carboxylic units derived from unsaturated mono- or dicarboxylic acid monomers of formula: 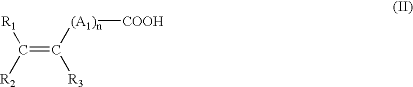
n is an integer from 0 to 10,
A₁ is chosen from a methylene group wherein, when n is greater than 1, at least one of said methylene groups can be bonded to a hetero atom, such as oxygen and sulphur,
R₁ is chosen from hydrogen, phenyl groups and benzyl groups,
R₂ is chosen from hydrogen, lower alkyls, and carboxyl groups,
R₃ is chosen from hydrogen, lower alkyls, —CH₂—COOH, phenyl groups, and benzyl groups; and
polymers comprising units derived from sulphonic acid.

7. The composition of claim 6, wherein said units derived from sulphonic acid are chosen from vinylsulphonic, styrenesulphonic, and acrylamidoalkylsulphonic units.

8. The composition of claim 1, wherein the cationic polymers are chosen from cationic proteins and the hydrolysates of cationic polymers are chosen from hydrolysates of cationic proteins.

9. The composition of claim 1 wherein the anionic polymers are chosen from:
acrylic acid copolymers;
copolymers derived from crotonic acid;
polymers derived from at least one monomer chosen from itaconic, fumaric and maleic acids and anhydrides and at least one monomer chosen from vinyl esters, vinyl ethers, vinyl halides, phenylvinyl derivatives, acrylic acid and its esters;
copolymers of methacrylic acid and of methyl methacrylate;
copolymers of methacrylic acid and of ethyl acrylate;
copolymers of crotonic acid and of vinyl acetate; and
vinyl acetate/crotonic acid/polyethylene glycol terpolymers.

10. The composition of claim 9, whereins said acrylic acid copolymers are chosen from the terpolymers acrylic acid/ethyl acrylate/N-tert-butylacrylamide

11. The composition of claim 9 wherein the esters of the acrylic acid are chosen from monoesterified maleic anhydride/methylvinyl ether copolymers.

12. The composition of claim 9 wherein the copolymers derived from crotonic acid are chosen from the terpolymers vinyl acetate/vinyl tert-butylbenzoate/crotonic acid and the terpolymers crotonic acid/vinyl acetate/vinyl neododecanoate.

13. The composition of claim 1, wherein the amphoteric polymers are chosen from polymers comprising units derived from:
a) at least one monomer chosen from acrylamides comprising an alkyl substituted nitrogen and methacrylamides comprising an alkyl substituted nitrogen;
b) at least one acidic comonomer comprising at least one reactive carboxyl group, and
c) at least one basic comonomer chosen from esters comprising at least one substiuent chosen from primary, secondary, tertiary and quaternary amine substituents of acrylic and methacrylic acids and the product of quaternization of dimethylaminoethyl methacrylate with a sulphate chosen from dimethyl and diethyl sulphate.

14. The composition of claim 1 wherein the nonionic polymers are chosen from:
polyalkyloxazolines;
vinyl acetate homopolymers;
acrylic ester and vinyl acetate copolymers;
ethylene and vinyl acetate copolymers;
copolymers of vinyl acetate and of maleic ester;
maleic anhydride and polyethylene copolymers;
homopolymers of alkyl acrylates and homopolymers of alkyl methacrylates;
copolymers of acrylic esters;
copolymers of acrylonitrile and of a nonionic monomer; and
copolymers of alkyl acrylate and urethane.

15. The composition of claim 14 wherein the copolymers of acrylic esters are chosen from copolymers of alkyl acrylates and alkyl methacrylates and wherein the nonionic monomer is chosen from butadiene and alkyl (meth)acrylates.

16. The composition of claim 1 wherein the cationic polymers are chosen from polymers comprising units in the principal polymer chain comprising at least one group chosen from primary, secondary, tertiary and quaternary amine groups and polymers comprising at least one side substituent directly linked to the principal polymer chain, wherein the at least one side substituent comprises at least one group chosen from primary, secondary, tertiary and quaternary amine groups.

17. The composition of claim 1 wherein said cationic polymers are chosen from cationic cyclocopolymers, cationic polysaccharides, and quaternary polymers of vinylpyrrolidone and vinylimidazole.

18. The composition of claim 17 wherein said cationic cyclopolymers are chosen from the homopolymers of diallyidimethylammonium chloride and the copolymers of diallyidimethylammonium chloride and acrylamide.

19. The composition of claim 17 wherein said cationic polysaccharides are chosen from guar gums modified with a 2,3-epoxypropyltrimethylammonium salt and quaternary cellulose ether derivatives,

20. The composition of claim 19 wherein said quaternary cellulose ether derivatives are chosen from polycondensates of hydroxyethylcelluloses with an epoxide substituted with a trimethylammonium group.

21. The composition of claim 1 wherein the quaternary ammonium salts comprising at least one amide functional group are chosen from the formula (XXI): 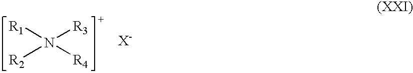
wherein
R₁ to R₄, which may be identical or different, are chosen from alkyl radicals comprising 1 to 30 carbon atoms, (C₂-C₆)polyoxyalkylene, (C₁₂-C₂₂)alkylamido(C₂-C₆)alkyl, (C₁₂-C₂₂)alkyl acetate, hydroxyalkyl radicals comprising from 1 to 30 carbon atoms; provided that at least one of R₁ to R₄ contains at least one amide functional group; and
X⁻ is an anion chosen from halides, phosphates, acetates, lactates, (C₂-C₆)alkyl sulphates, and alkyl and alkylaryl sulphonates.

22. The composition of claim 1 wherein the quaternary ammonium salts of imidazolinium are chosen from the formula (XXII): 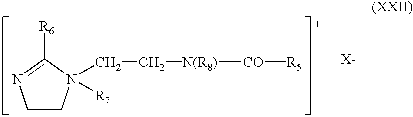
wherein
R₅ is chosen from alkenyl and alkyl radicals comprising from 8 to 30 carbon atoms,
R₆ is chosen from a hydrogen atom, C₁-C₄ alkyl radicals, and alkenyl and alkyl radicals comprising from 8 to 30 carbon atoms,
R₇ is chosen from alkyl radicals having from 1 to 4 carbon atoms;
R₈ is chosen from a hydrogen atom and 1-4 alkyl radicals; and
X⁻ is an anion chosen from halides, phosphates, acetates, lactates, alkyl sulphates, alkyl sulphonates and alkylaryl sulphonates.

23. The composition of claim 22 wherein the alkyl and alkenyl radicals of R₅ are derived from tallow fatty acids;

24. The composition of claim 1, wherein the quaternary diammonium salts are chosen from those of formula (XXIII): 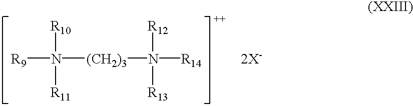
in which:
R₉ is chosen from aliphatic radicals comprising from about 16 to 30 carbon atoms;
R₁₀, R₁₁, R₁₂, R₁₃ and R₁₄, which are identical or different, are chosen from hydrogen and alkyl radicals comprising from 1 to 4 carbon atoms; and
X⁻ is an anion chosen from halides, acetates, phosphates, nitrates and methyl sulphates.

25. The composition of claim 24 in which the quaternary diammonium salts are chosen from propanetallowdiammonium dichloride.

26. The composition of claim 1 wherein the quaternary ammonium salts containing at least one ester functional group are chosen from the formula 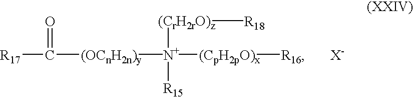
in which:
R₁₅ is chosen from linear and branched C₁-C₆ alkyl, C₁-C₆ hydroxyalkyl, and C₁-C₆ dihydroxyalkyl radicals;
R₁₆ is chosen from 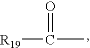
a hydrogen atom, saturated and unsaturated, linear and branched C₁-C₂₂ hydrocarbon radicals R₂₀;
R₁₈ is chosen from 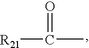
a hydrogen atom, saturated and unsaturated, linear and branched C₁-C₆ hydrocarbon radicals R₂₂;
R₁₇, R₁₉ and R₂₁ are independently chosen from saturated and unsaturated, linear and branched C₇-C₂₁ hydrocarbon radicals;
n, p and r, which are identical or different, are chosen from integers ranging from 2 to 6;
y is chosen from integers ranging from 1 to 10;
x and z, which are identical or different, are chosen from integers ranging from 0 to 10; and
X⁻ is an anion chosen from simple and complex, organic and inorganic anions;
provided, however, that the sum of x+y+z is equal to 1 to 15, that when x is equal to 0, then R₁₆ is R₂₀, and that when z is equal to 0, then R₁₈ is R₂₂.

27. The composition of claim 1 wherein the at least one anionic surfactant is present at a concentration relative to the total weight of the composition of from 1 to 30% by weight.

28. The composition of claim 1 wherein the at least one anionic surfactant is present at a concentration relative to the total weight of the composition of from 3 to 15% by weight.

29. The composition of claim 1 wherein the at least one soluble conditioning agent is present at a concentration relative to the total weight of the composition of from 0.001% to 20% by weight.

30. The composition of claim 1 wherein the at least one soluble conditioning agent is present at a concentration relative to the total weight of the composition of from 0.01% to 10% by weight.

31. The composition of claim 1 wherein the at least one soluble conditioning agent is present at a concentration relative to the total weight of the composition of from 0.05% to 3% by weight.

32. The composition of claim 1 further comprising at least one additional surfactant chosen from anionic, non-ionic, and amphoteric surfactants.

33. The composition of claim 32, wherein said at least one additional surfactant is present at a concentration relative to the total weight of the composition of from 0.1% to 40% by weight.

34. The composition of claim 32, wherein said at least one additional surfactant is present at a concentration relative to the total weight of the composition of from 3% to 30% by weight.

35. The composition of claim 32, wherein said at least one additional surfactant is present at a concentration relative to the total weight of the composition of from 5% to 20% by weight.

36. The composition of claim 1 further comprising at least one additive chosen from thickeners, perfumes, pearlescent agents, preservatives, sunscreens, silicones, ceramides, pseudoceramides, linear and branched chain C₁₆-C₄₀ acids, hydroxy acids, vitamins, provitamins, vegetable oils, mineral oils, synthetic oils and antidandruff agents.

37. The composition of claim 36 wherein said linear and branched chain C₁₆-C₄₀ acids comprise 18-methyleicosanoic acid.

38. The composition of claim 36 wherein said provitamins are chosen from panthenol.

39. A shampoo or washing composition for the skin, a rinse-off or leave-in after-shampoo, a composition for permanent waving, straightening, dyeing or bleaching, or a rinse-off composition for permanent waving, straightening, dyeing or bleaching comprising, in a cosmetically acceptable medium,
(a) at least one anionic surfactant chosen from N-acylated compounds of mono- and polyamidated polycarboxylic amino acids and the salts of said acids and
(b) at least one soluble conditioning agent chosen from:
anionic polymers,
nonionic polymers,
cationic polymers,
amphoteric polymers,
hydrolysates of cationic polymers, and
cationic surfactants chosen from quaternary ammonium salts comprising at least one amide functional group, quaternary ammonium salts of imidazoline, quaternary diammonium salts, quaternary ammonium salts comprising at least one ester functional group.

40. A method for washing a keratinous material comprising applying to said keratinous material an effective amount of a composition comprising, in a cosmetically acceptable medium,
(a) at least one anionic surfactant chosen from N-acylated compounds of mono- and polyamidated polycarboxylic amino acids and the salts of said acids and
(b) at least one soluble conditioning agent chosen from:
anionic polymers,
nonionic polymers,
cationic polymers,
amphoteric polymers,
hydrolysates of cationic polymers, and
cationic surfactants chosen from quaternary ammonium salts comprising at least one amide functional group, quaternary ammonium salts of imidazoline, quaternary diammonium salts, quaternary ammonium salts comprising at least one ester functional group.

41. A method for increasing the disentanglement or sleeking of hair comprising applying to the hair an effective amount of a composition comprising, in a cosmetically acceptable medium,
(a) at least one anionic surfactant chosen from N-acylated compounds of mono- and polyamidated polycarboxylic amino acids and the salts of said acids and
(b) at least one soluble conditioning agent chosen from:
anionic polymers,
nonionic polymers,
cationic polymers,
amphoteric polymers,
hydrolysates of cationic polymers, and
cationic surfactants chosen from quaternary ammonium salts comprising at least one amide functional group, quaternary ammonium salts of imidazoline, quaternary diammonium salts, quaternary ammonium salts comprising at least one ester functional group,
said applying occurring for a time sufficient to impart at least one of volume, lightness, softness, suppleness or manageability to the hair.

42. A method for treating a keratinous material comprising applying to said material an effective amount of a cosmetic composition and then optionally rinsing off, wherein said cosmetic composition comprises,
(a) at least one anionic surfactant chosen from N-acylated compounds of mono- and polyamidated polycarboxylic amino acids and the salts of said acids and
(b) at least one soluble conditioning agent chosen from:
anionic polymers,
nonionic polymers,
cationic polymers,
amphoteric polymers,
hydrolysates of cationic polymers, and
cationic surfactants chosen from quaternary ammonium salts comprising at least one amide functional group, quaternary ammonium salts of imidazoline, quaternary diammonium salts, quaternary ammonium salts comprising at least one ester functional group.

43. The method of claim 42 wherein said keratinous material is hair.
